(19) United States
(12) Patent Application Publication
(10) Pub. No.: US 2024/0138932 A1
(43) Pub. Date: May 2, 2024

(54) SYSTEMS AND METHODS FOR CONTROLLING ONE OR MORE SURGICAL TOOLS

(71) Applicant: Warsaw Orthopedic, Inc., Warsaw, IN (US)

(72) Inventors: Shane Lars Riding, Bartlett, TN (US); Julien J. Prevost, Memphis, TN (US); Jonathan M. Dewey, Memphis, TN (US); Gregory Thomas Poulter, Zionsville, IN (US)

(21) Appl. No.: 17/976,540

(22) Filed: Oct. 28, 2022

(51) Int. Cl. A61B 34/30; A61B 17/17; A61B 90/00

(52) U.S. Cl. CPC A61B 34/30; A61B 17/1728; A61B 90/03; A61B 2090/033

(57) ABSTRACT

Systems and methods for performing a surgical procedure to form a custom shaped cavity in an anatomical element are provided. A surgical tool may be oriented by a robotic arm in one direction along a first trajectory to remove a first portion of the anatomical element. The surgical tool may be oriented in at least one direction along a second trajectory to remove a second portion of the anatomical element to form a custom shaped cavity in the anatomical element.

### BACKGROUND

The present disclosure is generally directed to controlling one or more surgical tools, and relates more particularly to controlling one or more surgical tools using a surgical robot.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A system for performing a minimally-invasive surgical procedure according to at least one embodiment of the present disclosure comprises a processor; and a memory storing data for processing by the processor, the data, when processed, causes the processor to: receive a surgical tool through an arm guide of a robotic arm; and cause the robotic arm to orient the surgical tool in at least one direction along a trajectory to remove at least a portion of an anatomical element to form a custom shaped cavity in the anatomical element.

Any of the aspects herein, wherein the custom shaped cavity has cross-sectional area larger than a cross-sectional area of the surgical tool.

Any of the aspects herein, wherein the at least one direction comprises a lateral direction and a depth direction.

Any of the aspects herein, further comprising the arm guide, wherein the arm guide comprises a depth stop to prevent movement of the surgical tool past a predetermined depth, and wherein the depth stop is adjustable.

Any of the aspects herein, wherein the memory stores further data for processing by the processor that, when processed, causes the processor to: cause the robotic arm to stop movement of the surgical tool at a predetermined depth.

Any of the aspects herein, wherein the surgical tool is configured to drill and mill the anatomical element.

Any of the aspects herein, wherein the surgical tool comprises a reamer, and wherein the memory stores further data for processing by the processor that, when processed, causes the processor to: receive a drill bit through the arm guide of the robotic arm; and cause the robotic arm to orient the drill bit in one direction along a drill bit trajectory to form a bore in the anatomical element prior to removal of the at least the portion of the anatomical element by the reamer.

Any of the aspects herein, wherein the memory stores further data for processing by the processor that, when processed, causes the processor to: cause the robotic arm to orient the surgical tool in one direction along a drill bit trajectory to form a bore in the anatomical element prior to removal of the at least the portion of the anatomical element; measure a depth of the bore; and cause the robotic arm to drive an implant in the bore to the depth.

Any of the aspects herein, wherein the custom shaped cavity is shaped to seat a head of a pedicle screw.

Any of the aspects herein, wherein the custom shaped cavity is shaped such that the head is tilted relative to the pedicle screw.

Any of the aspects herein, wherein the surgical tool has a diameter larger than a cutting depth of the surgical tool.

A system for performing a surgical procedure according to at least one embodiment of the present disclosure comprises a robotic arm configured to orient a surgical tool; a processor; and a memory storing data for processing by the processor, the data, when processed, causes the processor to: cause the robotic arm to orient the surgical tool in at least one direction along a trajectory to remove at least a portion of an anatomical element to form a custom shaped cavity in the anatomical element.

Any of the aspects herein, wherein the surgical procedure is a minimally invasive surgical procedure.

Any of the aspects herein, wherein the custom shaped cavity has cross-sectional area larger than a cross-sectional area of the surgical tool.

Any of the aspects herein, wherein the at least one direction comprises a lateral direction and a depth direction.

Any of the aspects herein, further comprising an arm guide comprising a depth stop to prevent movement of the surgical tool past a predetermined depth, and wherein the depth stop is adjustable; and wherein the memory stores further data for processing by the processor that, when processed, causes the processor to: receive the surgical tool through the arm guide.

A system for performing a surgical procedure according to at least one embodiment of the present disclosure comprises a robotic arm configured to orient a surgical tool; an arm guide coupled to the robotic arm and configured to prevent movement of the surgical tool past a predetermined depth; a processor; and a memory storing data for processing by the processor, the data, when processed, causes the processor to: receive a first surgical tool through the arm guide; cause the robotic arm to orient the first surgical tool in one direction along first a trajectory to form a bore in the anatomical element; receive a second surgical tool through the arm guide; cause the robotic arm to orient the second surgical tool in more than one direction along a second trajectory to remove a second portion of the anatomical element to form a custom shaped cavity in the anatomical element.

Any of the aspects herein, wherein the first surgical tool comprises a drill bit and the second surgical tool comprises a reamer.

Any of the aspects herein, wherein the custom shaped cavity has cross-sectional area larger than a cross-sectional area of the surgical tool.

Any of the aspects herein, wherein the at least one direction comprises a lateral direction and a depth direction.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a block diagram of a system according to at least one embodiment of the present disclosure;

FIG. 3 is a flowchart according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In minimally invasive surgical (MIS) procedures, users such as surgeons are unable to utilize many of the conventional tools used in open surgeries as the working space in MIS procedures is much smaller than open surgeries. Further, users may have a difficult time viewing, or cannot view, the surgical space in an MIS procedure. In particular, procedures in which material is removed from an anatomical element such as decortication may prove challenging in an MIS procedure. With no visibility, small incisions, and the added risk of soft tissue contact, installation of implants such as pedicle screw implants using MIS procedures may be difficult. A further risk during MIS procedures is that the implants may be left proud due to the bony anatomy that cannot be removed without acceptable risk to the patient.

According to at least one embodiment of the present disclosure, a robotic surgical system can enable the ability to remove boney anatomy using a surgical tool such as reamer (or any other tool capable of removing the boney anatomy) with increased accuracy, while simultaneously minimizing risk to the patient. This would allow the user to remove boney anatomy along a multi-directional trajectory of the screw. Such removal combined with the accuracy of depth (also enabled by the robotic system) allows the user to ream or remove boney anatomy to a predetermined depth, place a depth marker, and then place a pedicle screw to that depth. This would allow MIS surgeries to be completed with greater ease and success.

Embodiments of the present disclosure provide technical solutions to one or more of the problems of (1) removing portion(s) of an anatomical element in an MIS procedure, (2) forming one or more custom shaped cavities in one or more anatomical elements in difficult to reach locations, (3) performing decortication in an MIS procedure, and (4) increasing safety to the patient.

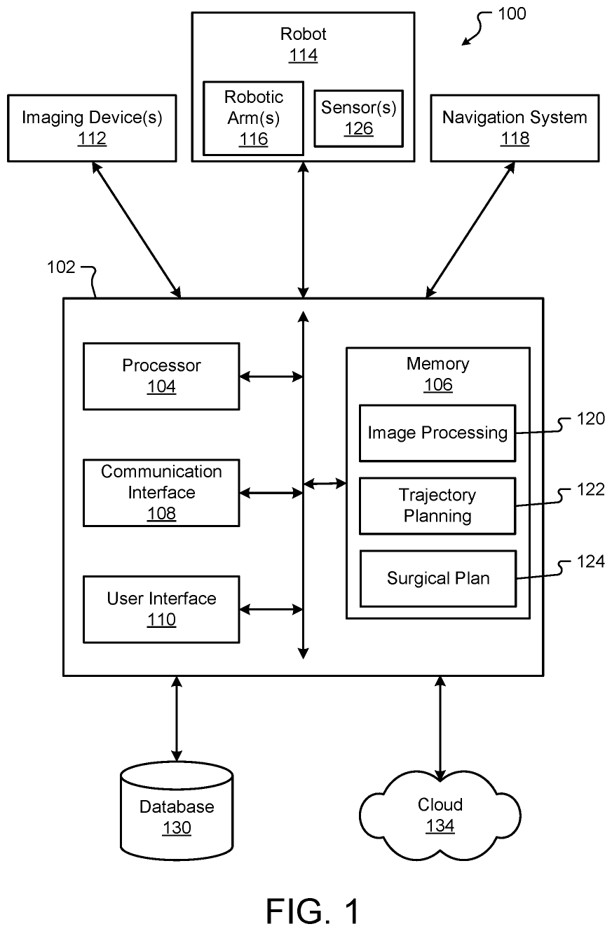

Turning first to FIG. 1, a block diagram of a system 100 according to at least one embodiment of the present disclosure is shown. The system 100 may be used to control one or more surgical tools using a robotic system, e.g., control, pose, and/or otherwise manipulate a surgical robotic system, a surgical robotic arm, and/or surgical tools attached thereto and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 300 and 400 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120 and/or trajectory planning 122. Such content, if provided as instructions, may, in some embodiments, be organized into one or more applications, modules, packages, layers, or engines.

The image processing 120 enables the processor 104 to process image data of an image (received from, for example, the imaging device 112, an imaging device of the navigation system 118, or any imaging device) for the purpose of, for example, identifying information about an anatomical element such as an anatomical element 236 and/or objects such as a surgical tool 234 depicted in the image. The information may comprise, for example, identification of hard tissue and/or soft tissues, a boundary between hard tissue and soft tissue, a boundary of hard tissue and/or soft tissue, identification of a surgical tool such as the surgical tool 234, etc. The image processing 120 may, for example, identify hard tissue, soft tissue, and/or a boundary of the hard tissue and/or soft tissue by determining a difference in or contrast between colors or grayscales of image pixels. For example, a boundary between the hard tissue and the soft tissue may be identified as a contrast between lighter pixels and darker pixels. The image processing 120 may also be used to obtain pose information of the surgical tool 234 and/or the anatomical element 236 for the purpose of, for example, confirming a pose of the surgical tool 234 as obtained from the robot 114 when the robot 114 is orienting the surgical tool 234. The image processing 120 may also be used to obtain one or more measurements of the anatomical element 236 such as, for example, a depth of a bore in the anatomical element.

The trajectory planning 122 enables the processor 104 to receive information about a desired custom shaped cavity and generate a trajectory for the surgical tool 234 to remove one or more portions from the anatomical element 236. The one or more portions removed may form a bore and/or the desired custom shaped cavity. The bore and the desired custom shaped cavity are connected in at least one some embodiments to receive, for example, a pedicle screw implant. The custom shaped cavity may be shaped to receive and seat a screw head of the pedicle screw implant. The information about the desired custom shaped cavity may include, for example, dimensions of a desired custom shaped cavity, a three-dimensional model of the desired custom shaped cavity, dimensions of an implant to which the desired custom shaped cavity is to receive, and/or a three-dimensional model of the implant. The trajectory may be transmitted directly to, for example, to the robot 114 and/or stored in the database 130, the memory 106, or any memory of any component.

The memory 106 may also store a surgical plan 124. The surgical plan 124 may comprise, for example, one or more steps for performing a surgical procedure and/or one or more parameters during the surgical procedure. In some embodiments, the surgical procedure may be a spinal procedure (e.g., a spinal alignment, installing implants, osteotomy, fusion, and/or any other spinal procedure) to correct a spinal deformity. For example, the surgical plan 124 may comprise one or more surgical steps for installing one or more implants such as preparing an anatomical element 236 (e.g., a vertebra), drilling the anatomical element 236, tapping the anatomical element 236, decortication of the anatomical element 236, and driving an implant into the anatomical element 236. The surgical plan 124 may also be stored in the database 130.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the surgical tool 234 at one or more precise position(s) and orientation(s) (whether based on guidance from the navigation system 118 or not), and/or to return the surgical tool 234 to the same position(s) and orientation(s) at a later point in time to accomplish or to assist with a surgical task. In some embodiments, the robot 114 may be configured to hold and/or manipulate an anatomical element such as the anatomical element 236 during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the surgical tool 234. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool 234, or other object held by the robot 114 (or, more specifically, by the robotic arm 116) may be precisely positionable in one or more needed and specific positions and orientations.

The robotic arm(s) 116 may comprise one or more sensors 126 that enable the processor 104 (or a processor of the robot 114) to determine a precise pose in space of the robotic arm (as well as any object or element held by or secured to the robotic arm).

The sensor 126 may be a position sensor, a proximity sensor, a magnetometer, or an accelerometer. In some embodiments, the sensor 126 may be a linear encoder, a rotary encoder, or an incremental encoder. In still other embodiments, the sensor 126 may be an imaging sensor. Other types of sensors may also be used as the sensor 126. For example, the sensor 126 may be a force sensor, configured to detect a force applied on the robotic arm 116 (e.g., whether via an end effector of the robotic arm 116, a tool held by an end effector of the robotic arm 116, or otherwise). The one or more sensors 126 may be positioned, for example, on the robotic arm 116 or elsewhere.

Data from the sensor(s) 126 may be provided to a processor of the robot 114, to the processor 104 of the computing device 102, and/or to the navigation system 118. The data may be used to calculate a position in space of the robotic arm 116 relative to one or more coordinate systems (e.g., based on coordinate system information stored in the memory 116). The calculation may be based not just on data received from the sensor(s) 126, but also on data or information (such as, for example, physical dimensions) about, for example, the robot 114 or a portion thereof, or any other relevant object, which data or information may be stored, for example, in a memory 116 of a computing device 102 or in any other memory.

In some embodiments, reference markers (e.g., navigation markers) may be placed on the robot 114 (including, e.g., on the robotic arm 116), the imaging device 112, the surgical tool 234, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools 234 (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, the one or more surgical plans 124 (including, for example, information about a desired custom shaped cavity, a trajectory for the surgical tool 234, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 300 and/or 400 described herein. The system 100 or similar systems may also be used for other purposes.

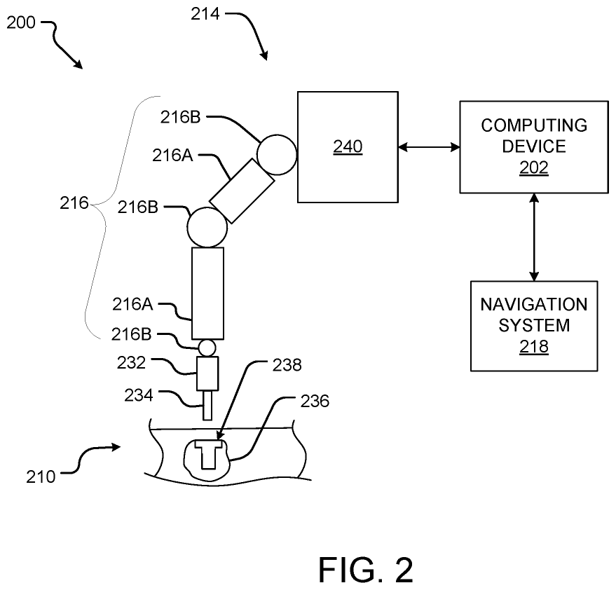

Turning to FIG. 2 a block diagram of a system 200 according to at least one embodiment of the present disclosure is shown. The system 200 includes a computing device 202 (which may be the same as or similar to the computing device 102 described above), a navigation system 218 (which may be the same as or similar to the navigation system 118 described above), and a robot 214 (which may be the same as or similar to the robot 114 described above). The system 200 may be used with the system 100 in some embodiments. Systems according to other embodiments of the present disclosure may comprise more or fewer components than the system 200. For example, the system 200 may not include the navigation system 218.

As illustrated, the robot 214 includes a robotic arm 216 (which may comprise one or more members 216A connected by one or more joints 216B) extending from a base 240. The base 240 may be stationary or movable. In some embodiments, the robot 214 may include one robotic arm or two or more robotic arms. In embodiments where the robot 214 includes more than two robotic arms, the robotic arms may operate in a shared or common coordinate space. By operating in the common coordinate space, the robotic arms avoid colliding with each other during use, as a position of each robotic arm is known to each other.

In some embodiments, an arm guide 232 and the surgical tool 234 may be disposed or supported on an end of the robotic arm 216. In other embodiments, the arm guide 232 and the surgical tool 234 be disposed or secured to any portion of the robotic arm 216. In other embodiments, any one or more tool(s), instrument(s), or component(s) may be supported by, secured to, or disposed on a robotic arm.

The arm guide 232 may be attached to the robotic arm 216 and may be configured to receive one or more surgical tools 234. In some embodiments, the arm guide 232 may comprise an eyelet through which the surgical tool 234 is received through. The arm guide 232 may also comprise a step or a depth stop that may interface with the surgical tool 234 to prevent the surgical tool 234 from moving past the depth stop. The depth stop may act as a physical barrier to prevent the surgical tool 234 from moving past a predetermined depth. In some embodiments, the depth stop may be a component separate from the arm guide 232. In such embodiments, the depth stop may be manually controlled or set by a user such as, for example, a surgeon or a medical provider, or automatically by the robot 114 and/or the robotic arm 116. It will also be appreciated that the robotic arm 216 may also automatically prevent the surgical tool 234 from moving past the predetermined threshold. Thus, the surgical tool 234 may be prevented from moving past the predetermined threshold mechanically and/or using software.

As illustrated in FIG. 2, the surgical tool 234 is supported by the robotic arm 216 and the arm guide 232. The robotic arm 216 is operable to execute one or more planned movements and/or procedures using the surgical tool 234 autonomously and/or based on input from a surgeon or user. In other words, the surgical tool 234 may be used to perform an action or a procedure on a patient 210, whether based on instructions from a surgeon and/or pursuant to a surgical plan such as the surgical plan 124. For example, the surgical tool 234 may be used to install an implant in the patient 210 by performing one or more steps such as preparing the anatomical element 236 (e.g., a vertebra), drilling the anatomical element 236 to form a bore, tapping the anatomical element 236, decortication of the anatomical element 236, and driving an implant into the anatomical element 236.

The surgical tool 234 may comprise, for example, a drill bit, a reamer, a screwdriver, a drill tap, a combination drill bit-reamer capable of drilling, reaming, and/or milling, and/or any other surgical tool. In embodiments where the surgical tool 234 comprises a reamer, the reamer may be a tool with a flat face. More specifically, the reamer may be a tool with face having a width great than a depth of cut. The surgical tool 234 may also be shaped to avoid anatomy or hardware. For example, the surgical tool 234 may comprise a cutter with a portion of the middle of a body of the cutter removed such that after placing, for example, a pedicle screw shank, the cutter (which may be used to clear bone around the head of the pedicle screw) and the head can be received inside a pocket formed by the cutter. The pocket, which may be a central pocket and/or a fully cannulated pocket, can be in fluid communication with a source of fluid such as, for example, water, saline or other flushing fluid, which may be used to flush the surgical site and/or suction surgical debris. The cutter may also be combined with another surgical tool 234 such as, for example, a drill capable of perforating the anatomical element (which may comprise, for example, a pedicle) thereby allowing one surgical tool 234 to perform multiple tasks

In another example, the surgical tool 234 may comprise a cutting tool that can be smaller than a desired pocket and can be used to cut multiple smaller pockets that are proximate to the cavity 238. More specifically, in some embodiments a plunged pocket can be overlapped such that the overlapping forms a larger pocket than the size of the cutting tool. In such embodiments, non-standard or custom-shaped counterbores can be formed with a standard tool by overlapping the cuttings into the desired shape. More specifically, for example, overlapping two cylindrical counterbores may form an oblong pocket, and these overlaps can be combined into a desired or custom shape. In at least one embodiment, the shape of the pocket (which may be the same as the cavity 238 or adjacent or proximal to the cavity 238) may be be tailored to the desired implant, such that, for example, a long rod could be received into and elongate pocket. Further, the custom cavity 238, pocket, or any other custom shaped bone removal may be programmed using software whether automatically (e.g., using artificial algorithms) or manually (e.g., using user input from, for example, a surgeon or other medical provider). The software may receive input such as, for example, coordinates for a desired cavity, a desired shape of bone removal appropriate for an implant, and/or maximizing or optimizing the strength of the bone (e.g., either as measured through bone density or a thickness of remaining bone after removal the bone removal).

Decortication of the anatomical element 236 may include using the surgical tool 234 to remove at least a portion of the anatomical element 236 to form a custom shaped cavity 238. The custom shaped cavity 238 may include a complex volume and may have multiple portions of different cross-sectional areas. In some embodiments, the robotic arm 216 may be manipulated with the surgical tool 234 attached thereto to create a multi-portioned custom shaped cavity 238. In other embodiments, the robotic arm 216 may be static during use (e.g., the robotic arm 216 is in a fixed positioned) and the surgical tool 234 may be used in conjunction with the robotic arm 216. The upper-most portion of the custom shaped cavity 238 may have a first shape and a first cross-sectional area, a middle or lower portion of the custom shaped cavity 238 may have a second shape and a second cross-sectional area. In some embodiments, the cross-sectional area of the upper-most portion is larger than the cross-sectional area of the middle or lower portion. Providing a multi-portioned custom shaped cavity 238 can help to accommodate implants having a complex shape, and may further prevent such implants from projecting too far outside (e.g., proud) of the anatomical element 236 in which the implant is secured.

A custom shaped cavity 238 can also be formed at an angle relative to a pedicle screw shank such that as a pedicle screw head is attached to the pedicle screw shank, the head interferes with the anatomical element (e.g., bone) and is tilted relative to the pedicle screw shank. In such instances, the tilted head can be used for additional correction of a patient's spine, such that as a rod is mated with the head, the head may orient itself perpendicular to the rod, thereby orienting the anatomical element 236 (e.g., vertebral body) with it. It may also be desirable to have the head tilted at an angle relative to the pedicle screw shank such that the custom shaped cavity 238 may leave more anatomical element 236 (e.g., bone) remaining than a conventional, in-line pocket. Such custom shaped cavity 238 may potentially result in increased strength in, for example, a direction of the pedicle screw pull-out from the anatomical element (e.g., bone). In other instances, the cavity 238 may be shaped to enable, for example, medial-lateral pivoting of the head while restricting an inferior-superior pivoting of the head.

Such custom shaped cavity 238 is enabled by the robotic arm 216 precisely operating the surgical tool 234 in a working volume that may not be visible to a user such as the surgeon or other medical provider. In other words, the robotic arm 216 is capable of orienting and operating the surgical tool 234 in spaces that the user cannot view particularly in, for example, MIS procedures. Thus, cavities of complex geometries or shapes may be formed by the robotic arm 216 operating the surgical tool 234 in a working volume that is conventionally difficult to work within.

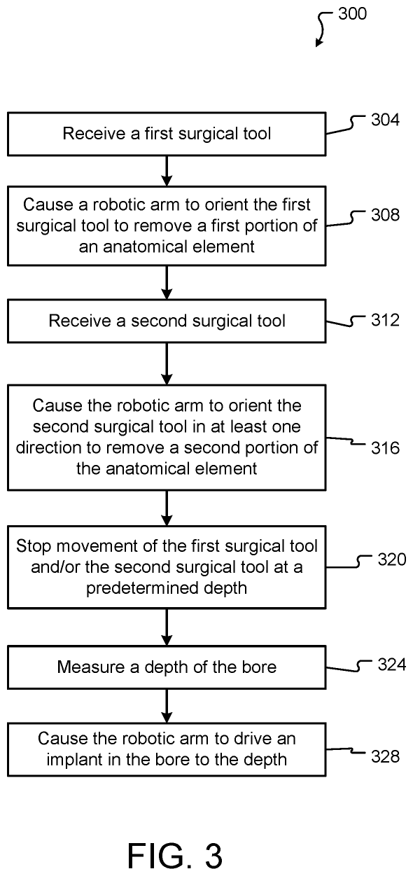

FIG. 3 depicts a method 300 that may be used, for example, for controlling one or more surgical tools such as the surgical tool 234 to form a custom shaped cavity in an anatomical element such as the anatomical element 236. Though the method 300 will be described relative to installing an implant such as a pedicle screw implant, it will be appreciated that the method 300 can be used to form a custom-shaped cavity for any purpose, reason, or use.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as an image processing 120 and/or a trajectory planning 122.

The method 300 comprises receiving a first surgical tool (step 304). The first surgical tool may be the same as or similar to the surgical tool 234. In some embodiments, the first surgical tool comprises a drill bit. In other embodiments, the first surgical tool comprises a combination drill bit-reamer or any tool capable of drilling, reaming, and/or milling. The first surgical tool may be received by and coupled to, for example, a robotic arm such as the robotic arm 116, 216 of a robot such as the robot 114, 214. In some embodiments, an arm guide such as the arm guide 232 may be attached to the robotic arm and the first surgical tool may be received through the arm guide. In such embodiments, the arm guide may be configured to receive the first surgical tool (or any surgical tool) and prevent movement of the surgical tool past a predetermined depth. Whether the first surgical tool is received by the arm guide or is otherwise coupled to the robotic arm, the first surgical tool is supported, oriented, and operated by the robotic arm. As previously described, the robotic arm is capable of precisely positioning and orienting a surgical tool such as the first surgical tool and is configured to orient the surgical tool along a trajectory.

The method 300 also comprises causing a robotic arm to orient the first surgical tool to remove a first portion of an anatomical element (step 308). Causing the robotic arm to orient the first surgical tool may include causing the robotic arm to orient the first surgical tool in one direction along a first trajectory to remove the first portion of the anatomical element, which may be the same as or similar to the anatomical element 236. The first portion, in some embodiments, forms a bore in the anatomical element. In such embodiments, the first trajectory may comprise a depth for the first surgical tool to drill to in the anatomical element. The first trajectory may be obtained from, for example, a memory such as the memory 106 of a computing device such as the computing device 102, a surgical plan such as the surgical plan 124, and/or a database such as the database 130. In other instances, the first trajectory may be obtained from, for example, the processor executing a trajectory planning such as the trajectory planning 122. As previously described, the trajectory planning enables the processor to receive information about a desired custom shaped cavity and/or a desired bore and generate a trajectory for the surgical tool to remove one or more portions from the anatomical element. The one or more portions may form the bore, a custom shaped cavity, or any other shape, volume, or combinations of shapes.

The method 300 also comprises receiving a second surgical tool (step 312). The step 312 may be the same as or similar to the step 304 described above. The second surgical tool may comprise a reamer. In some embodiments, the second surgical tool may be the same tool as the first surgical tool. In such embodiments, the method 300 may not include the step 312. In other words, the robotic arm (or the arm guide) may receive the surgical tool and perform both the step 308 and the step 316 below.

The method 300 also comprises causing the robotic arm to orient the second surgical tool in at least one direction to remove a second portion of the anatomical element (step 316). The step 316 may be the same as or similar to the step 308. The at least one direction may comprise a lateral direction and depth direction. It will be appreciated that the at least one direction may comprise any direction(s). The second portion removed from the anatomical element may form a custom shaped cavity such as the custom shaped cavity 238 in the anatomical element. The custom shaped cavity in some instances may have a cross-sectional area larger than a cross-sectional area of the second surgical tool (or any surgical tool). In some embodiments, the custom shaped cavity may be shaped to receive a head of a pedicle screw implant such that the head may seat flush against the anatomical element. The custom shaped cavity enables the pedicle screw implant to have a lower profile relative to the anatomical element. Without such custom shaped cavity, the head and the pedicle screw may be left proud and exposed relative to the anatomical element.

In some embodiments, the step 316 may be referred to as a decortication step. Decortication of the anatomical element may include using the surgical tool to remove at least a portion of the anatomical element to form the custom shaped cavity. Such decortication is enabled by the robotic arm precisely operating the surgical tool in a working volume that may not be visible to a user such as the surgeon or other medical provider. In other words, the robotic arm is capable of orienting and operating the surgical tool in spaces that the user cannot view particularly in, for example, MIS procedures. Thus, cavities of complex geometries or shapes may be formed by the robotic arm operating the surgical tool in a working volume that is conventionally difficult to work within.

The method 300 also comprises stopping movement of the first surgical tool and/or the second surgical tool at a predetermined depth (step 320). The predetermined depth may be received from, for example, the surgical plan, the memory, the database, or any other component of a system such as the system 100. In embodiments where the arm guide is attached to the robotic arm and the surgical tool (whether the first surgical tool, the second surgical tool, or any surgical tool) is received by the arm guide, the arm guide may be configured to stop movement of the surgical tool at the predetermined depth. More specifically, the arm guide may comprise a step or a depth stop that may interface with the surgical tool to prevent the surgical tool from moving past the depth stop. Thus, the depth stop acts as a physical barrier to prevent the surgical tool from moving past a predetermined depth. The depth stop may be adjustable such that a user such as a surgeon or other medical provider can adjust the depth stop as needed. In other embodiments, the robotic arm 216 may also automatically prevent the surgical tool from moving past the predetermined depth. In such embodiments, the robotic arm may receive the predetermined depth and automatically stop movement of the surgical tool past the predetermined depth.

The method 300 also comprises measuring a depth of the bore (step 324). The depth of the bore may be measured using image data from an imaging device such as the imaging device 112 and/or using the robotic arm to measure the depth. In embodiments where imaging is used to measure the depth, image data from the imaging device may be processed by the processor using image processing such as the image processing 120 to process the image data to identify the bore and measure a depth of the bore. In embodiments where the robotic arm is used to measure the depth, a sensor such as the sensor 126 of the robotic arm may be used to measure the depth. For example, a pose of the robotic arm may be measured when the first surgical tool beings drilling and another pose of the robotic arm when the first surgical tool has drilled to the desired depth. In another example, the pose of the robotic arm may be measured when the first surgical tool is at the desired depth.

The depth can also be received from the surgical plan or determined when the processor executes the trajectory planning. The depth as received from the surgical plan, the trajectory planning, or any software may be used to confirm or compare to the depth as measured by the robotic arm and/or the imaging device. Such comparison may be used to determine an accuracy of the depth.

The method 300 also comprises causing the robotic arm to drive an implant in the bore to the depth (step 328). The depth may be obtained from, for example, the step 324. In other embodiments, the depth may be marked using a depth marker and a navigation system such as the navigation system 118 configured to track a pose of the depth marker. In still other embodiments, the depth may be received from the surgical plan, the memory, the database, or any other component.

Causing the robotic arm to drive the implant (such as a pedicle screw implant) into the bore may include the robotic arm using a surgical tool such as a screwdriver to drive the implant. It will be appreciated that the steps 308, 316, and/or 328 (e.g., drilling the bore, forming the custom-shaped cavity, and driving the implant) may be performed with the same surgical tool. It will also be appreciated that the method 300 may include other steps such as, for example, preparing the anatomical element and tapping the anatomical element and in such instances any combination of steps may be performed with the same surgical tool.

The present disclosure encompasses embodiments of the method 300 that comprise more or fewer steps than those described above, and/or one or more steps that are different than the steps described above.

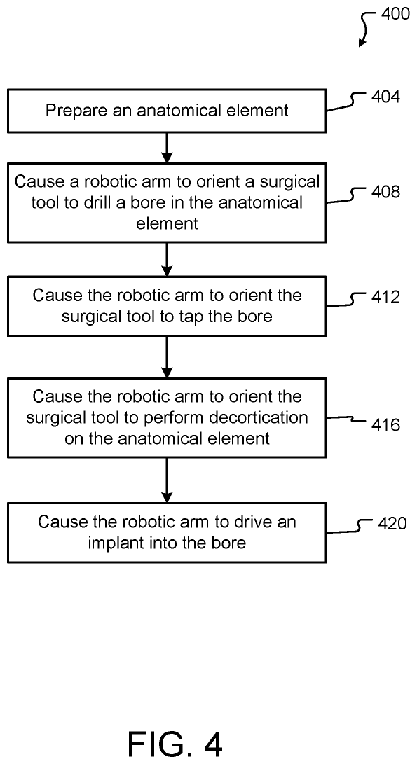

FIG. 4 depicts a method 400 that may be used, for example, for controlling one or more surgical tools such as the surgical tool 234 to form prepare an anatomical element such as the anatomical element 236 for receiving an implant such as the implant 238. Though the method 400 will be described relative to installing an implant such as a pedicle screw implant, it will be appreciated that the method 400 can be used to form a custom-shaped cavity for any purpose, reason, or use.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as an image processing 120 and/or a trajectory planning 122.

The method 400 comprises preparing an anatomical element (step 404). The anatomical element may be the same as or similar to the anatomical element 236 and may comprise, for example, a vertebra. The anatomical element may be prepared to receive, for example, an implant such as the implant 238. The implant may be a pedicle screw, though in other instances the implant may be, for example, a rod, a cage, etc. Preparing the anatomical element may include scraping, polishing, marking, forming a pilot hole, or a combination thereof on a surface of the anatomical element. A surgical tool such as the surgical tool 234 may be used to prepare the anatomical element. The anatomical element may be prepared manually by a user operating the surgical tool or by a robotic arm such as the robotic 116, 216 of a robot such as the robot 114, 214 assisting the user or automatically orienting and operating the surgical tool. In some embodiments, an arm guide such as the arm guide 232 may be attached to the robotic arm and the surgical tool may be received through the arm guide. In such embodiments, the arm guide may be configured to receive the surgical tool (or any surgical tool) and prevent movement of the surgical tool past a predetermined depth. Whether the surgical tool is received by the arm guide or is otherwise coupled to the robotic arm, the surgical tool is supported, oriented, and operated by the robotic arm. As previously described, the robotic arm is capable of precisely positioning and orienting a surgical tool such as the surgical tool and is configured to orient the surgical tool along a trajectory.

The method 400 also comprises causing the robotic arm to orient the surgical tool to drill a bore in the anatomical element (step 408). The surgical tool may be the same as or similar to the surgical tool used in the step 404 to prepare the anatomical element. In other instances, the surgical tool may be a different surgical tool than the surgical tool used in the step 404. Causing the robotic arm to orient the surgical tool may include causing the robotic arm to orient the surgical tool in one direction along a first trajectory to remove the first portion of the anatomical element (e.g., drill). The first portion, in some embodiments, forms the bore in the anatomical element. In such embodiments, the first trajectory may comprise a depth for the surgical tool to drill to in the anatomical element. As previously described, the first trajectory may be obtained from, for example, a memory such as the memory 106 of a computing device such as the computing device 102, a surgical plan such as the surgical plan 124, and/or a database such as the database 130. In other instances, the first trajectory may be obtained from, for example, the processor executing a trajectory planning such as the trajectory planning 122. As previously described, the trajectory planning enables the processor to receive information about a desired custom shaped cavity and/or a desired bore and generate a trajectory for the surgical tool to remove one or more portions from the anatomical element. The one or more portions may form the bore, a custom shaped cavity, or any other shape, volume, or combinations of shapes.

The method 400 also comprises causing the robotic arm to orient the surgical tool to tap the bore (step 412). The surgical tool may be the same as or similar to the surgical tool used in the steps 404 and/or 408 to prepare the anatomical element and/or to drill the anatomical element. In other instances, the surgical tool may be a different surgical tool than the surgical tool used in the steps 404 and/or 408. Causing the robotic arm to orient the surgical tool may include causing the robotic arm to orient the surgical tool in one direction along the first trajectory to tap the bore formed in the step 408.

The method 400 also comprises causing the robotic arm to orient the surgical tool to perform decortication on the anatomical element (step 416). The step 416 may be the same as or similar to the step 316 of the method 300 described above. The surgical tool may be the same as or similar to the surgical tool used in the steps 404, 408, and/or 412 to prepare the anatomical element, to drill the anatomical element, and/or to tap the bore. In other instances, the surgical tool may be a different surgical tool than the surgical tool used in the steps 404, 408, and/or 412. Orienting the surgical tool to perform decortication may include orienting the surgical tool in at least one direction to remove a second portion of the anatomical element The at least one direction may comprise a lateral direction and depth direction. It will be appreciated that the at least one direction may comprise any direction(s). The second portion removed from the anatomical element may form a custom shaped cavity such as the custom shaped cavity 238 in the anatomical element. The custom shaped cavity in some instances may have a cross-sectional area larger than a cross-sectional area of the second surgical tool (or any surgical tool). In some embodiments, the custom shaped cavity may be shaped to receive a head of a pedicle screw implant such that the head may seat flush against the anatomical element. The custom shaped cavity enables the pedicle screw implant to have a lower profile relative to the anatomical element. Without such custom shaped cavity, the head and the pedicle screw may be left proud and exposed relative to the anatomical element.

As previously described, decortication of the anatomical element may include using the surgical tool to remove at least a portion of the anatomical element to form the custom shaped cavity. Such decortication is enabled by the robotic arm precisely operating the surgical tool in a working volume that may not be visible to a user such as the surgeon or other medical provider. In other words, the robotic arm is capable of orienting and operating the surgical tool in spaces that the user cannot view particularly in, for example, MIS procedures. Thus, cavities of complex geometries or shapes may be formed by the robotic arm operating the surgical tool in a working volume that is conventionally difficult to work within.

The method 400 also comprises causing the robotic arm to drive an implant into the bore (step 420). The step 420 may be the same as or similar to the step 328 of the method 300 described above. Causing the robotic arm to drive the implant (such as a pedicle screw implant) into the bore may include the robotic arm using a surgical tool such as a screwdriver to drive the implant. It will be appreciated that the steps 404, 408, 412, 416, and/or 420 (e.g., preparing the anatomical element, drilling a bore in the anatomical element, tapping the bore, forming the custom-shaped cavity (e.g., decortication), and driving the implant) may be performed with the same surgical tool. It will also be appreciated that the method 400 may include other steps such as, for example, preparing the anatomical element and tapping the anatomical element and in such instances any combination of steps may be performed with the same surgical tool.

The present disclosure encompasses embodiments of the method 400 that comprise more or fewer steps than those described above, and/or one or more steps that are different than the steps described above.

As noted above, the present disclosure encompasses methods with fewer than all of the steps identified in FIGS. 3 and 4 (and the corresponding description of the methods 300 and 400), as well as methods that include additional steps beyond those identified in FIGS. 3 and 4 (and the corresponding description of the methods 300 and 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for performing a minimally-invasive surgical procedure comprising:
a processor; and
a memory storing data for processing by the processor, the data, when processed, causes the processor to:
receive a surgical tool through an arm guide of a robotic arm; and
cause the robotic arm to orient the surgical tool in at least one direction along a trajectory to remove at least a portion of an anatomical element to form a custom shaped cavity in the anatomical element.

2. The system of claim 1, wherein the custom shaped cavity has cross-sectional area larger than a cross-sectional area of the surgical tool.

3. The system of claim 1, wherein the at least one direction comprises a lateral direction and a depth direction.

4. The system of claim 1, further comprising the arm guide, wherein the arm guide comprises a depth stop to prevent movement of the surgical tool past a predetermined depth, and wherein the depth stop is adjustable.

5. The system of claim 1, wherein the memory stores further data for processing by the processor that, when processed, causes the processor to:
cause the robotic arm to stop movement of the surgical tool at a predetermined depth.

6. The system of claim 1, wherein the surgical tool is configured to drill and mill the anatomical element.

7. The system of claim 1, wherein the surgical tool comprises a reamer, and wherein the memory stores further data for processing by the processor that, when processed, causes the processor to:
receive a drill bit through the arm guide of the robotic arm; and
cause the robotic arm to orient the drill bit in one direction along a drill bit trajectory to form a bore in the anatomical element prior to removal of the at least the portion of the anatomical element by the reamer.

8. The system of claim 1, wherein the memory stores further data for processing by the processor that, when processed, causes the processor to:
cause the robotic arm to orient the surgical tool in one direction along a drill bit trajectory to form a bore in the anatomical element prior to removal of the at least the portion of the anatomical element;
measure a depth of the bore; and
cause the robotic arm to drive an implant in the bore to the depth.

9. The system of claim 1, wherein the custom shaped cavity is shaped to seat a head of a pedicle screw.

10. The system of claim 9, wherein the custom shaped cavity is shaped such that the head is tilted relative to the pedicle screw.

11. The system of claim 1, wherein the surgical tool has a diameter larger than a cutting depth of the surgical tool.

12. A system for performing a surgical procedure comprising:
a robotic arm configured to orient a surgical tool;
a processor; and
a memory storing data for processing by the processor, the data, when processed, causes the processor to:
cause the robotic arm to orient the surgical tool in at least one direction along a trajectory to remove at least a portion of an anatomical element to form a custom shaped cavity in the anatomical element.

13. The system of claim 12, wherein the surgical procedure is a minimally invasive surgical procedure.

14. The system of claim 12, wherein the custom shaped cavity has cross-sectional area larger than a cross-sectional area of the surgical tool.

15. The system of claim 12, wherein the at least one direction comprises a lateral direction and a depth direction.

16. The system of claim 12, further comprising an arm guide comprising a depth stop to prevent movement of the surgical tool past a predetermined depth, and wherein the depth stop is adjustable; and wherein the memory stores further data for processing by the processor that, when processed, causes the processor to:
receive the surgical tool through the arm guide.

17. A system for performing a surgical procedure comprising:
a robotic arm configured to orient a surgical tool;
an arm guide coupled to the robotic arm and configured to prevent movement of the surgical tool past a predetermined depth;
a processor; and
a memory storing data for processing by the processor, the data, when processed, causes the processor to:
receive a first surgical tool through the arm guide;
cause the robotic arm to orient the first surgical tool in one direction along first a trajectory to form a bore in the anatomical element;
receive a second surgical tool through the arm guide;
cause the robotic arm to orient the second surgical tool in more than one direction along a second trajectory to remove a second portion of the anatomical element to form a custom shaped cavity in the anatomical element.

18. The system of claim 17, wherein the first surgical tool comprises a drill bit and the second surgical tool comprises a reamer.

19. The system of claim 17, wherein the custom shaped cavity has cross-sectional area larger than a cross-sectional area of the surgical tool.

20. The system of claim 17, wherein the at least one direction comprises a lateral direction and a depth direction.
